(19) United States
(12) Patent Application Publication
(10) Pub. No.: US 2012/0203084 A1
(43) Pub. Date: Aug. 9, 2012

(54) POSITIONING SYSTEM, APPARATUS, AND METHOD FOR WIRELESS MONITORING OF ESOPHAGEAL pH VALUE

(75) Inventors: Xiangdong LI, Chongqing (CN); Jian YUAN, Chongqing (CN); Lang QIN, Chongqing (CN); Wanli TONG, Chongqing (CN); Zhaotao GONG, Chongqing (CN)

(73) Assignee: Chongqing Jinshan Scinece & Technology (Group) Co., Ltd., Chongqing (CN)

(21) Appl. No.: 13/447,322

(22) Filed: Apr. 16, 2012

Related U.S. Application Data
(63) Continuation-in-part of application No. PCT/CN2010/001623, filed on Oct. 15, 2010.

(30) Foreign Application Priority Data
Oct. 16, 2009 (CN) 200910191195.1

(51) Int. Cl. A61B 5/145

(52) U.S. Cl. 600/309

(57) ABSTRACT

A positioning system, an apparatus, and a method for wireless monitoring of esophageal pH value. The positioning system includes an internal transmitting apparatus and an external recording apparatus. The external recording apparatus includes a second element, and the second element of the external recording apparatus cooperates with a first element of the internal transmitting apparatus. If the external recording apparatus detects that the internal transmitting apparatus is not located in the preset region, alerting action is conducted under the control of a micro-processor. The external recording apparatus periodically detects the intensity of a signal received by a second wireless transceiver module, under the control of the micro-processor, and if the signal intensity is not within the preset range, the alerting action is conducted under the control of the micro-processor.

### CROSS-REFERENCE TO RELATED APPLICATIONS

This application is a continuation-in-part of International Patent Application No. PCT/CN2010/001623 with an international filing date of Oct. 15, 2010, designating the United States, now pending, and further claims priority benefits to Chinese Patent Application No. 200910191195.1 filed Oct. 16, 2009. The contents of all of the aforementioned applications, including any intervening amendments thereto, are incorporated herein by reference.

### CORRESPONDENCE ADDRESS

Inquiries from the public to applicants or assignees concerning this document should be directed to: MATTHIAS SCHOLL P. C., ATTN.: DR. MATTHIAS SCHOLL ESQ., 14781 MEMORIAL DRIVE, SUITE 1319, HOUSTON, Tex. 77079.

### BACKGROUND OF THE INVENTION

1. Field of the Invention

The invention relates to a positioning system, apparatus, and method for wireless monitoring of esophageal pH value.

2. Description of the Related Art

Conventional medical apparatuses for esophageal diagnosis and treatment include, for example, a push-type upper digestive tract endoscope, such as gastroscope, electronic gastroscope, and ultrasonic endoscope, which enters the esophagus, observes the lesion area, acquires the image, and conducts resection. However, it is not suitable for a long-period operation, as the tolerance of patients to the above-mentioned endoscopes is poor. Optionally, a catheter-type physiological parameter monitor is employed for esophageal diagnosis and treatment, such as catheter-type pH meter, catheter-type manometer, and catheter-type bilirubin meter. However, these meters require an indwelling catheter, which brings pains to patients, makes patients painful and unable to eat, and it is also difficult for long time use.

Recently, a variety of radio telemetries have been invented and applied. A capsule-shaped internal miniature device is swallowed and moves with the digestive peristalsis, acquires the image, and detects the parameters of the digestive tract such as pH value and pressure. Data are transmitted to an external miniature receiver through radio frequency. However, the capsule-shaped internal miniature device is unable to be fixed, and thus it cannot monitor specific three-dimensional space for a long time.

Currently, in the market, there is a system for wireless monitoring of esophageal pH value, including a pH capsule, a data recorder, an analysis software, and a fixture. For the system, a pin is used to fix the pH capsule on the esophageal wall, and the recorded pH data are wirelessly transmitted to a receiver at the waist of patient, so that there is no catheter electrode indwelled. To some extent, the system overcomes the above mentioned shortcomings

However, the system has the following shortcomings:
first, it is unable to know the fixing condition of the pH capsule in real time, and thus the invalid detection due to the unexpected drop of the capsule cannot be avoided absolutely, thereby increasing the cost of monitoring, meanwhile, due to the unexpected drop of the capsule, it is also unable to ensure the testing accuracy of the pH capsule;
second, as the testing of the system usually lasts for 24-48 hours, it is inevitable for the patients to change the position of the data recorder when they are working or sleeping, which may interrupt signal and affect the integrity of testing;
furthermore, as the storage time and environmental temperature vary, the testing accuracy of the pH sensor may vary, which affects the testing accuracy.

### SUMMARY OF THE INVENTION

In view of the above-described problem, it is one object of the invention to provide a position system for wireless monitoring of esophageal pH value, it is another object of the invention to provide an apparatus for wireless monitoring of esophageal pH value, and it is further an object of the invention to provide a method for wireless monitoring of esophageal pH value, which can avoid the invalid detection due to the unexpected drop of the capsule and reduce the detection cost.

To achieve the above objectives, in accordance with one embodiment of the invention, there provided is a positioning system for wireless monitoring of esophageal pH value, comprising: an internal transmitting apparatus, and an external recording apparatus; wherein the internal transmitting apparatus comprises a pH sensor, a sample circuit, a first micro-processor, a power management unit, a first wireless transceiver module, and a first element; the external recording apparatus comprises a second micro-processor, a power management unit, a buzzer, a memory, a data interface, a key, a second wireless transceiver module, a status indicator, a housing, and a second element; the second element of the external recording apparatus cooperates with the first element of the internal transmitting apparatus; if the external recording apparatus detects that the internal transmitting apparatus does not locate in a preset region, the micro-processor controls the buzzer and/or status indicator to alert; the external recording apparatus periodically detects the intensity of a signal received by second wireless transceiver module, under the control of the micro-processor, and if the signal intensity is not within a preset range, the micro-processor controls the buzzer and/or the status indicator to alert.

In a class of this embodiment, the first element of the internal transmitting apparatus is a permanent magnet; the second element of the external recording apparatus is a magnetic sensor; the second element of the external recording apparatus cooperates with the first element of the internal transmitting apparatus; the intensity of magnetic field generated by the permanent magnet of the internal transmitting apparatus is detected through the magnetic sensor, if the intensity of the magnetic field is not within a preset range, the external recording apparatus detects and records that the internal transmitting apparatus does not locate in the preset region.

In a class of this embodiment, the first element of the internal transmitting apparatus is a reed switch disposed in series between the pH sensor and the sample circuit; the second element of the external recording apparatus is a magnet, which actuates the reed switch through magnetic induction; the second element of the external recording apparatus cooperates with the first element of the internal transmitting apparatus; if the actual distance between the magnet and the reed switch is longer than a preset distance, the reed switch is actuated to open the circuit between the pH sensor and the sample circuit of the internal transmitting apparatus, and the external recording apparatus detects and records that the internal transmitting apparatus does not locate in the preset region.

In a class of this embodiment, the external recording apparatus further comprises a temperature sensor; the memory pre-stores the first pH calibration data of the internal transmitting apparatus; the temperature sensor detects the current room temperature, and sends the temperature data to the second micro-processor; the second wireless transceiver module receives the initial data from the internal transmitting apparatus and sends the data to the second micro-processor; the second micro-processor calibrates the initial data, and conducts temperature compensation in the course of calibration, to obtain the current calibration data; the second micro-processor compares the current calibration data with the first pH calibration data pre-stored in the memory; if the current calibration data differ from the first pH calibration data, a calibration alerting signal is sent to the internal transmitting apparatus through second wireless transceiver module; the internal transmitting apparatus further comprises a work light; the first wireless transceiver module of the internal transmitting apparatus receives the calibration alerting signal and sends the signal to the first micro-processor; and the first micro-processor controls the work light to alert.

In accordance with another embodiment of the invention, there provided is an internal transmitting apparatus, comprising a pH sensor, a sample circuit, a first micro-processor, a power management unit, and a first wireless transceiver module; wherein the pH sensor, the sample circuit, the first micro-processor, and the first wireless transceiver module are successively connected together; the power management unit is separately connected with the pH sensor, the sample circuit, the first micro-processor, and the first wireless transceiver module; the sample circuit, the first micro-processor, power management unit, and the first transceiver module are enclosed in a capsule housing; and a sensing portion of the pH sensor is exposed outside the capsule housing, and contacts with the body fluid of the esophagus; the internal transmitting apparatus further comprises a first element, which is disposed inside the capsule housing.

In a class of this embodiment, the first element is a permanent magnet, or a reed switch disposed in series between the pH sensor and the sample circuit.

In a class of this embodiment, the internal transmitting apparatus further comprises a work light, which is connected with the first micro-processor and receives the control signal from the first micro-processor to alert.

In accordance with a further embodiment of the invention, there provided is an external recording apparatus, comprising a second micro-processor, a power management unit, a buzzer, a memory, a data interface, a key, a second wireless transceiver module, a status indicator, a housing, and a second element; wherein the second element cooperates with the first element of the internal transmitting apparatus; if the internal transmitting apparatus 30 does not locate in a preset region, the second micro-processor controls the buzzer and/or the status indicator to alert; the external recording apparatus periodically detects the intensity of a signal received by the second transceiver module under the control of the second micro-processor, if the signal intensity is not within the preset range, the second micro-processor controls the buzzer and/or the status indicator to alert.

In a class of this embodiment, the external recording apparatus further comprises a temperature sensor; the memory pre-stores first pH calibration data of the internal transmitting apparatus; the temperature sensor detects the current room temperature, and sends the temperature data to the second micro-processor; the second wireless transceiver module receives the initial data from the internal transmitting apparatus and sends the data to the second micro-processor; the second micro-processor calibrates the initial data, and conducts temperature compensation in the course of calibration, to obtain the current calibration data; the second micro-processor compares the current calibration data with the first pH calibration data pre-stored in the memory; if the current calibration data differ from the first pH calibration data, a calibration alerting signal is sent to the internal transmitting apparatus through the second wireless transceiver module.

In accordance with a further embodiment of the invention, there provided is a method for wireless monitoring of esophageal pH value, comprising: determining if the internal transmitting apparatus locates in a preset region, through the cooperation between the recording apparatus and the internal transmitting apparatus, and alerting if the internal transmitting apparatus does not locate in the preset region; and determining if the signal intensity is within a preset range, based on the intensity of the received signal, which is periodically detected by the external recording apparatus, and alerting if the signal intensity is not within the preset range.

In a class of this embodiment, the method, before the system is put in use, further comprises: pre-storing the first pH calibration data of the internal transmitting apparatus in the external recording apparatus; calibrating the initial data received by the external recording apparatus from the internal transmitting apparatus, and conducting temperature compensation in the course of calibration on the initial data to obtain the current calibrated data; comparing the current calibrated data with the pre-stored first pH calibration data, and sending a calibration alerting signal to the internal transmitting apparatus if the current calibrated data differ from the first pH calibration data; and controlling the internal transmitting apparatus to alert after the internal transmitting apparatus receives the calibration alerting signal.

Advantages of the invention are summarized below. In the positioning system, apparatus, and method for wireless monitoring of esophageal pH value, the cooperation between the first element of the internal transmitting apparatus and the second element of the external recording apparatus enables the real time monitoring of the position of the internal transmitting apparatus in the esophagus, which not only avoids the invalid detection due to unexpected drop of the capsule, but also reduces the cost of detection for patients; in addition, the fact that the internal transmitting apparatus is applied for detecting the intensity of the signal received may help to avoid signal interrupt problem caused when the patients are working or sleeping, which ensures the integrality of detection data; furthermore, the calibration of the system before being put in use and the temperature compensation in the course of calibration improve the testing accuracy. Compared with the prior art, this invention has the advantages such as quicker acquisition of the positioning condition, more stability of signal, and more accuracy of detection, as well as ease of implementation, which is more acceptable to doctors and patients.

### BRIEF DESCRIPTION OF THE DRAWINGS

FIG. 1 is a schematic diagram of a positioning system in use according to one embodiment of the invention;

FIG. 2 is a circuit block diagram of an internal transmitting apparatus according to one embodiment of the invention;

FIG. 3 is a circuit block diagram of an external recording apparatus according to one embodiment of the invention;

FIG. 4 is a circuit block diagram of a second wireless transceiver module of the embodiment as shown in FIG. 3;

FIG. 5 is a flow diagram of positioning an internal transmitting apparatus according to one embodiment of the invention;

FIG. 6 is a flow diagram of alerting for communication failure according to one embodiment of the invention; and

FIG. 7 is a flow diagram of calibration of a positioning system prior to use according to one embodiment of the invention.

### DETAILED DESCRIPTION OF THE EMBODIMENTS

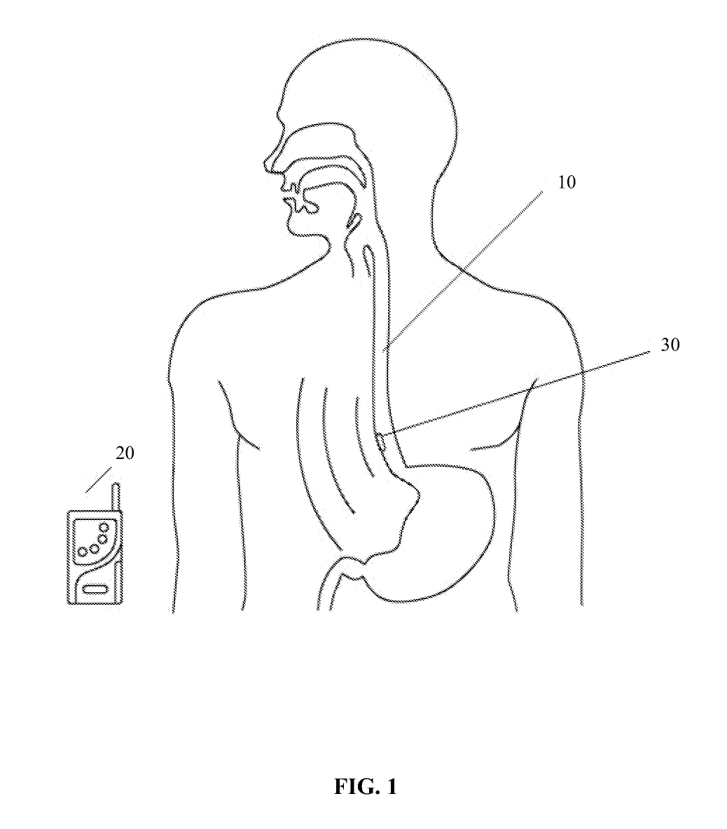

In the first embodiment of this invention, as shown in FIG. 1, an internal transmitting apparatus 30 is fixed on the esophagus 10, and data are transmitted between the internal transmitting apparatus 30 and an external recording apparatus 20 through radio frequency technology. The internal transmitting apparatus 30 is a pH capsule, and the pH capsule is streamlined and in a flat capsule-shaped structure. The external recording apparatus 20 is a data recorder.

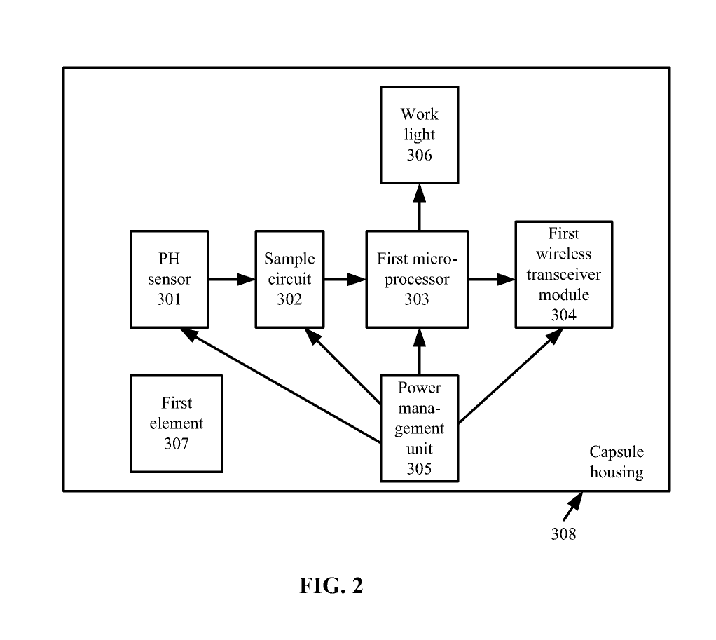

In FIG. 2, the internal transmitting apparatus 30 of this embodiment is in the form of a pH capsule. The pH capsule comprises a pH sensor 301, a sample circuit 302, a first micro-processor 303, a power management unit 305, and a first wireless transceiver module 304. The pH sensor 301, the sample circuit 302, the first micro-processor 303, and the first wireless transceiver module 304 are successively connected together. The power management unit 305 is separately connected with the pH sensor 301, the sample circuit 302, the first micro-processor 303, and the first wireless transceiver module 304. The sample circuit 302, the first micro-processor 303, power management unit 305, and the first transceiver module 304 are enclosed in a capsule housing 308. A sensing portion of the pH sensor 301 is exposed outside the capsule housing 308, and may contact with the body fluid in the esophagus. The pH capsule further comprises a first element 307, disposed inside the capsule housing 308.

As shown in FIG. 2, under the control of the first micro-processor 303, the pH sensor 301 detects the pH value of the body fluid in the esophagus periodically, and the pH value is converted into digital data through the sample circuit 302 and stored temporarily in the first micro-processor 303 of the capsule. After a certain period of time, the data packages are transmitted to an external data recorder through the first wireless transceiver module 304. Specifically, the external data recorder is the external recording apparatus 20. The power management unit 305 is a 3 V silver oxide button cell; the first micro-processor 303 is a chip with A/D unit and RAM built in. The pH sensor comprises a medical antimony measuring electrode and an Ag/AgCl reference electrode. The sample circuit 302 conducts impedance matching, signal amplification, and signal filtering. Thereafter, the built-in A/D unit of the first micro-processor 303 acquires the data, and then the data are transmitted to the external data recorder through the first wireless transceiver module 304 using FSK/ASK communication technology and 433 MHz ISM European band. The first wireless transceiver module 304 comprises a power amplifier (PA).

Specifically, in the embodiment as shown in FIG. 2, the first element 307 is a permanent magnet, which does not contact any component in the capsule housing 308. The material of the permanent magnet can be NdFeB, AlNiCo, or other high magnetism materials; the permanent magnet is in the shape of a sheet structure, and the direction of magnetization of the permanent magnet is in the width direction.

In addition, in the embodiment as shown in FIG. 2, the system further comprises a work light 306 connected with the first micro-processor 303, which alerts after receiving the control signal from the first micro-processor 303.

Furthermore, in other embodiments of this invention, the first element 307 is a reed switch disposed in series between the pH sensor 301 and the sample circuit 302.

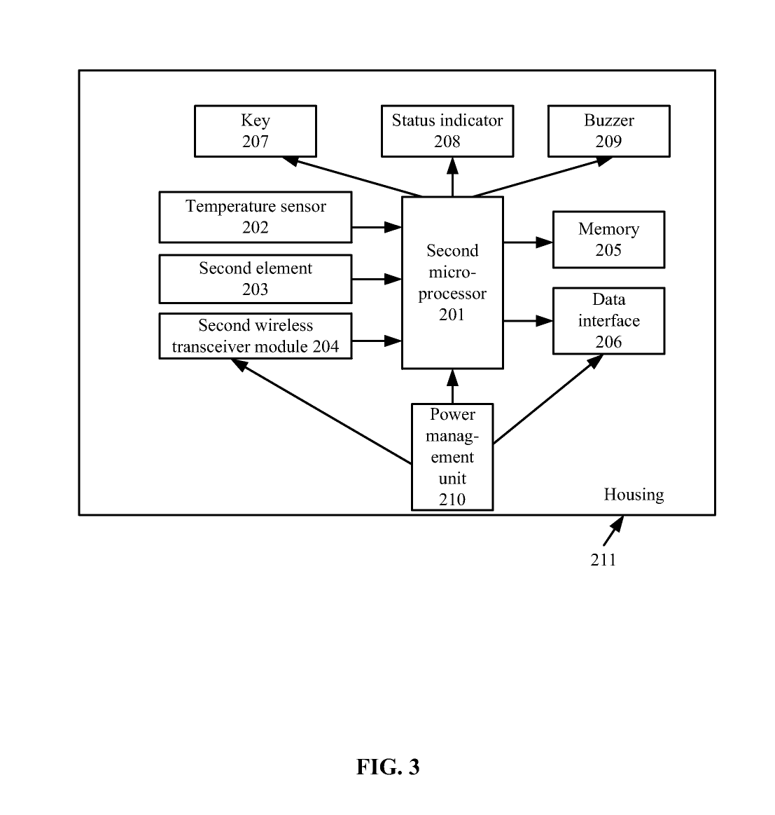

In the embodiment as shown in FIG. 3, a circuit block diagram of the external recording apparatus. The external recording apparatus is a data recorder. The data recorder comprises a second micro-processor 201, a power management unit 210, a buzzer 209, a memory 205, a data interface 206, a key 207, a second wireless transceiver module 204, a status indicator 208, a housing 211, and a second element 203; the above mentioned components are installed inside the housing 211 except the key 207. The second wireless transceiver module 204 receives pH data from the pH capsule 30, and the pH data are temporarily stored in the memory 205, or exported through the data interface 206, under the control of the second micro-processor 201. The computer controls the calibration of time and pH value of the data recorder through the data interface 206. The housing 211 of the data recorder is made of nontoxic materials; the patient may use the key 207 to record the status such as eating, sleeping, lying, and cardialgia. The memory 205 of the data recorder stores the pH data, and the pH data can be transmitted to the data processor such as the computer via the data interface 16. The power management unit 210 can be three No. 7 Alkali dry batteries. The memory 205 is nonvolatile memory such as Flash, Fram, and EEPROM. The status indicator 208 is in the form of red, green, yellow LED or other displaying components.

The second element 203 as shown in FIG. 3 cooperates with the first element 307 of the internal transmitting apparatus 30; if the internal transmitting apparatus 30 locates in the preset region, the data are sent by the internal transmitting apparatus 30 through the first wireless transceiver module 304; if the internal transmitting apparatus 30 does not locate in the preset region, the second micro-processor 201 controls the buzzer 209 and/or the status indicator 208 to alert.

More specifically, the first element 307 of the internal transmitting apparatus 30 is in the form of a permanent magnet, and the second element 203 of the external recording apparatus 20 is in the form of a magnetic sensor for the detection of the magnetic field of the permanent magnet; and the cooperation between the second element 203 and the first element 307 of the internal transmitting apparatus 30 refers to the fact that if the intensity of the magnetic field is within the preset range, the external recording apparatus 20 detects that the internal transmitting apparatus 30 locates in the preset region; and if the intensity of the magnetic field is not within the preset range, the external recording apparatus 20 detects that the internal transmitting apparatus 30 does not locate in the preset region.

Optionally, the first element 307 of the internal transmitting apparatus 30 is in the form of a reed switch, and the second element 203 of the external recording apparatus 20 is in the form of a magnet; the reed switch is actuated through magnetic induction; the cooperation between the second element 203 of the external recording apparatus and the first element 30 of the internal transmitting apparatus 30 refers to the fact that if the actual distance between the magnet and the reed switch is not longer than the preset distance, the reed switch is actuated to close the circuit between the pH sensor 301 and the sample circuit 302 of the internal transmitting apparatus 30, and the external recording apparatus 20 detects and records that the internal transmitting apparatus 30 locates in the preset region; if the actual distance between the magnet and the reed switch is longer than the preset distance, the reed switch is actuated to open the circuit between the pH sensor 301 and the sample circuit 302 of the internal transmitting apparatus 30, and the external recording apparatus 20 detects and records that the internal transmitting apparatus 30 does not locate in the preset region.

In addition, the external recording apparatus 20 periodically detects the intensity of the signal received by the second wireless transceiver module 204, under the control of the second micro-processor 201, and if the signal intensity is not within the preset range, the second micro-processor 201 controls the buzzer 209 and/or the status indicator 208 to alert.

Furthermore, the external recording apparatus 20 further comprises a temperature sensor 202; and the memory 205 pre-stores the first pH calibration data of the internal transmitting apparatus 30; and the temperature sensor 202 detects the current room temperature, and sends the temperature data to the second micro-processor 201; and the second wireless transceiver module 204 receives the initial data from the internal transmitting apparatus 30 and sends the data to the second micro-processor 201; and the initial data can be the voltage difference.

The second micro-processor 201 calibrates the initial data, and conducts temperature compensation in the course of calibration, to obtain the current calibration data; after that the second micro-processor 201 compares the current calibration data with the first pH calibration data pre-stored in the memory 205; if the current calibration data are the same as the first pH calibration data, the subsequent procedure is followed; if the current calibration data differ from the first pH calibration data, a calibration alerting signal is sent to the internal transmitting apparatus 30 through the second wireless transceiver module 204. And then the first wireless transceiver module 304 of the internal transmitting apparatus 30 receives the calibration alerting signal and sends the signal to the first micro-processor 303; and the first micro-processor 303 controls the work light 306 to alert.

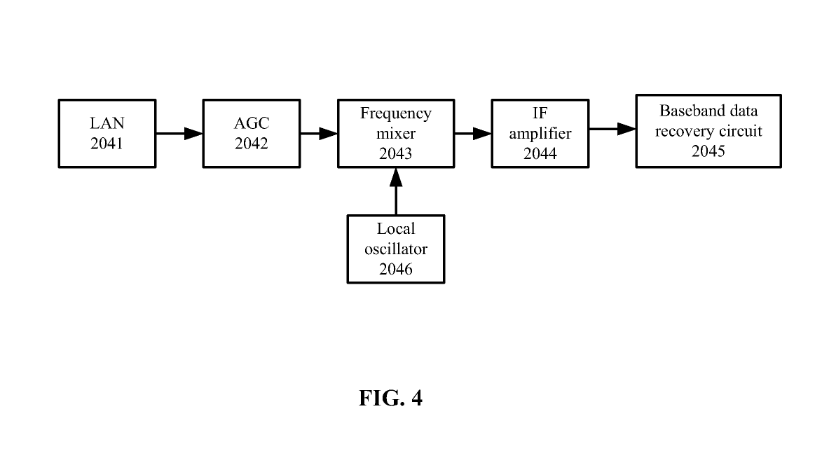

The second wireless transceiver module in FIG. 4 comprises a low noise amplifier LNA 2041, an automatic gain control circuit AGC 2042, a frequency mixer 2043, a local oscillator 2046, an IF amplifier 2044, and a baseband data recovery circuit 2045. The low noise amplifier 2041 may amplify the weak signal, so as to facilitate signal receiving thereafter; the AGC circuit 2042 can automatically adjust the gain of LNA 39 in terms of the detection of the signal intensity, and receive the signal with broader band; and the frequency mixer 2043 can be adopted to produce intermediate-frequency signal by mixing the frequency of the external high-frequency signal and that of the local signal, so as to facilitate data demodulation thereafter; the local oscillator 2046 comprises a PLL circuit, which synthesizes the local crystal oscillator signal into the signal with the frequency required by the frequency mixer 2043; the IF amplifier 2044 may preferably be in the form of an intermediate-frequency filter amplifier, which processes the intermediate-frequency signal produced by the frequency mixer 2043, so as to facilitate the data demodulation thereafter; the baseband data recovery circuit 2045 may preferably comprise a detecting circuit, a data filtering circuit, and a data shaping and recovery circuit, which demodulates low-frequency Analog signal.

With reference to FIG. 2 and FIG. 3, the second embodiment of this invention provides a positioning system for wireless monitoring of esophageal pH value comprising an internal transmitting apparatus 30, and an external recording apparatus 20. The internal transmitting apparatus 30 comprises a pH sensor 301, a sample circuit 302, a first micro-processor 303, a power management unit 305, a first wireless transceiver module 304, and a first element 307. The external recording apparatus 20 comprises a second micro-processor 201, a power management unit 210, a buzzer 209, a memory 205, a data interface 206, a key 207, a second wireless transceiver module 204, a status indicator 208, a housing 211, and a second element 203.

The second element 203 of the external recording apparatus 20 cooperates with the first element 307 of the internal transmitting apparatus 30; and if the external recording apparatus 20 detects that the internal transmitting apparatus 30 does not locate in the preset region, the second micro-processor 201 controls the buzzer 209 and/or status indicator 208 to alert.

The external recording apparatus 20 periodically detects the intensity of the signal received by second wireless transceiver module 204, under the control of the second micro-processor 201, and if the signal intensity is not within the preset range, the micro-processor 201 controls the buzzer 209 and/or the status indicator 208 to alert.

The first element 307 of the internal transmitting apparatus 30 is a permanent magnet; and the second element 203 of the external recording apparatus 20 is a magnetic sensor; and the cooperation between the second element 203 of the external recording apparatus 20 and the first element 307 of the internal transmitting apparatus 30 comprises the fact that the intensity of magnetic field generated by the permanent magnet of the internal transmitting apparatus is detected through the magnetic sensor, and if the intensity of magnetic field is not within the preset range, the external recording apparatus 20 detects that the internal transmitting apparatus 30 does not locate in the preset region.

The first element 307 of the internal transmitting apparatus 30 is a reed switch disposed in series between the pH sensor 301 and the sample circuit 302; and the second element 203 of the external recording apparatus 20 is a magnet, which actuates the reed switch through magnetic induction; the cooperation between the second element 203 of the external recording apparatus 20 and the first element 30 of the internal transmitting apparatus 30 specifically refers to the fact that if the actual distance between the magnet and the reed switch is no longer than the preset distance, the reed switch is actuated to close the circuit between the pH sensor 301 and the sample circuit 302 of the internal transmitting apparatus 30, and the external recording apparatus 20 detects that the internal transmitting apparatus 30 locates in the preset region; if the actual distance between the magnet and the reed switch is longer than the preset distance, the reed switch is actuated to open the circuit between the pH sensor 301 and the sample circuit 302 of the internal transmitting apparatus 30, and the external recording apparatus 20 detects that the internal transmitting apparatus 30 does not locate in the preset region.

Furthermore, the external recording apparatus 20 further comprises a temperature sensor 202; the memory 205 pre-stores the first pH calibration data of the internal transmitting apparatus 30; the temperature sensor 202 detects the current room temperature, and sends the temperature data to the second micro-processor 201; the second wireless transceiver module 204 receives the initial data from the internal transmitting apparatus 30 and sends the data to the second micro-processor 201. The initial data can be the voltage difference.

The second micro-processor 201 calibrates the initial data, and conducts temperature compensation in the course of calibration, to obtain the current calibration data; after that the second micro-processor 201 compares the current calibration data with the first pH calibration data pre-stored in the memory 205; if the current calibration data are the same as the first pH calibration data, the subsequent procedure is followed; if the current calibration data differ from the first pH calibration data, a calibration alerting signal is sent to the internal transmitting apparatus 30 through the second wireless transceiver module 204.

In addition, the data recorder may alert through the buzzer and/or the status indicator 208 under the control of the second micro-process 201.

The internal transmitting apparatus 30 further comprises a work light 306; and the first wireless transceiver module 304 of the internal transmitting apparatus 30 receives the calibration alerting signal and sends the signal to the first micro-processor 303; and then the first micro-processor 303 controls the work light 306 to alert.

The positioning system described in the embodiments of this invention enables the real time monitoring of the position of the internal transmitting apparatus in the esophagus through the cooperation between the first element 307 of the internal transmitting apparatus 30 and the second element of the external recording apparatus 20, which not only avoids the invalid detection due to unexpected drop of the capsule, but also reduces the cost of detection for patients; in addition, the fact that the internal transmitting apparatus 30 is applied for detecting the intensity of the received signal may help to avoid signal interrupt problem caused when the patients are working or sleeping, which ensures the integrality of detection data; furthermore, the calibration of the system before being put in use and the temperature compensation in the course of calibration improve the testing accuracy.

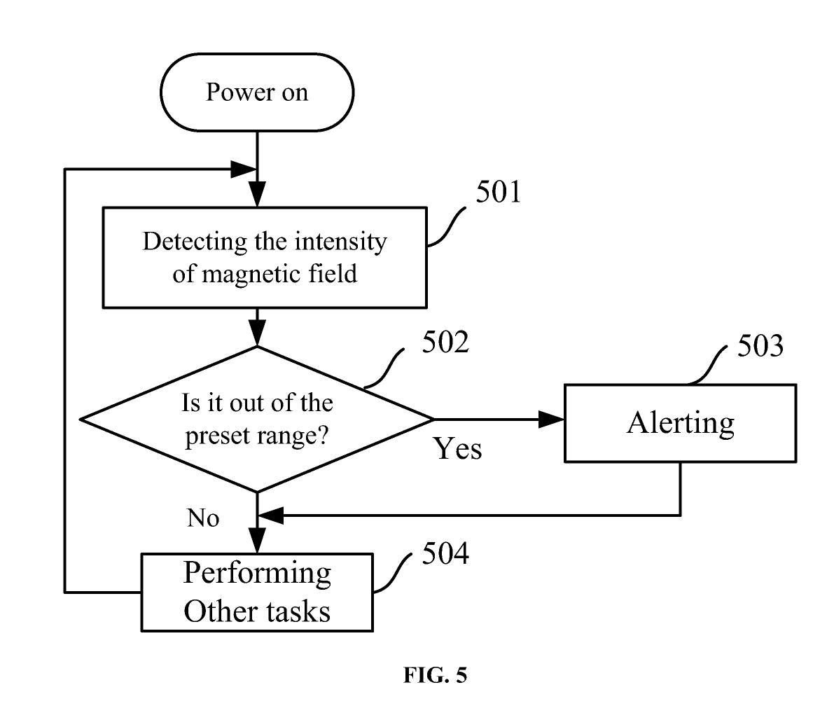

The FIG. 5 illustrates a flow diagram of positioning the internal transmitting apparatus. In the embodiment, the internal transmitting apparatus is a pH capsule, the first element of the internal transmitting apparatus is a permanent magnet, the external recording apparatus is a data recorder, and the second element of the external recording apparatus is a magnetic sensor; the procedure of positioning comprises:
step 501: allowing the data recorder to periodically detect the intensity of magnetic field generated by the permanent magnet in the capsule through the magnetic sensor 203 under the control of the second micro-processor 201;
step 502: determining if the intensity of the detected magnetic field of the capsule is within the preset range; and if it is within the preset range, performing step 503, otherwise, performing step 504;
step 503: allowing the data recorder to alert through the buzzer 209 and/or the status indicator 208, under the control of the second micro-processor 201, so as to indicate the failure of positioning;
step 504: ending the procedure, and performing other tasks.

The other tasks of the system in this invention refer to the tasks other than the abovementioned task, and the phrase will be also used in the following embodiments of the specification with the same meaning.

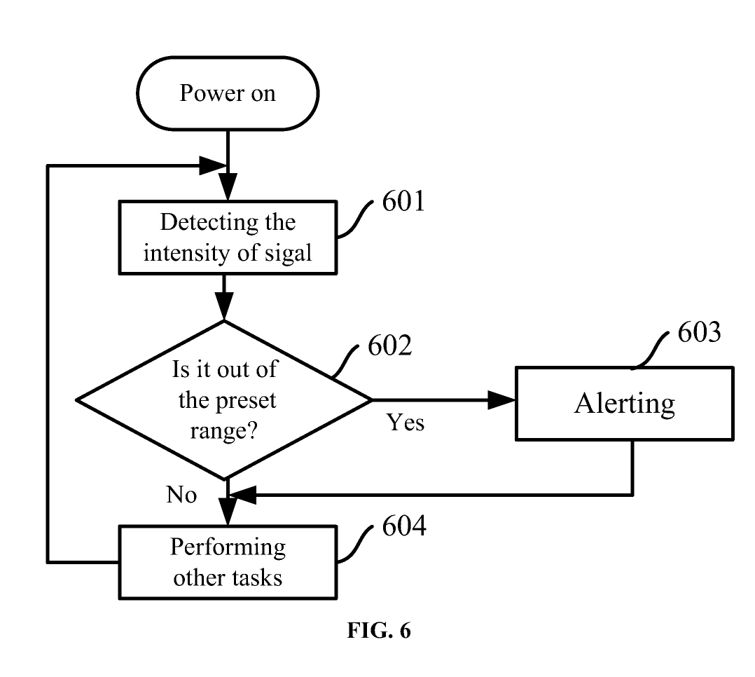

The FIG. 6 illustrates a flow diagram of alerting of communication failure; and in the embodiment of the invention, the internal transmitting apparatus is a pH capsule, and the external recording apparatus is a data recorder.

The procedure of alerting of communication failure comprises:
step 601: allowing the data recorder to periodically detect the intensity of a signal received by the second transceiver module 204, under the control of the second micro-processor 201;
step 602: determining if the detected intensity of the signal is within the preset range; if it is within the preset range, performing step 604, otherwise, performing step 603;
step 603: allowing the data recorder to alert through the buzzer 209 and/or the status indicator 208, under the control of the second micro-processor 201, so as to indicate the signal intensity failure;
step 604: ending the procedure, and performing other tasks.

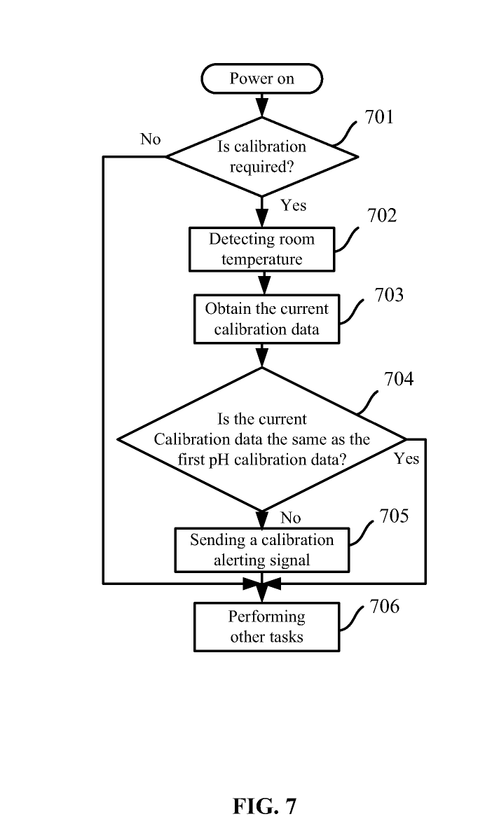

The FIG. 7 illustrates a flow diagram of calibration of the positioning system prior to use; and in the embodiment of the invention, the internal transmitting apparatus is a pH capsule, and the external recording apparatus is a data recorder.

The procedure of calibration comprises:
step 701: determining if there is a need for calibration in terms of the instruction received; if it is needed, performing step 702, otherwise, performing step 706;
step 702: detecting the room temperature through the temperature sensor 202 in the course of calibration;
step 703: receiving the initial data from the internal transmitting apparatus 30, calibrating the initial data, and conducting temperature compensation in the course of the calibration, to obtain the current calibration data; the temperature compensation comprising detecting the room temperature, and conducting temperature compensation for the calibrated pH-mV curve by conducting table lookup and processing with software, so as to minimize the influence of temperature on the testing.
step 704: determining the validity of the current calibration data, more specifically, which refers to determining if the current calibration data is the same as the first pH calibration data pre-stored; if they are the same, performing step 706, otherwise, performing step 705;
step 705: sending the calibration alerting signal to the internal transmitting apparatus; and meanwhile, allowing the data recorder to alert through the buzzer 209 and/or status indicator 208, under the control of the second micro-processor 201; for example, the buzzer sounds every 1 second, in order to notify the operator;
step 706: ending the procedure, and performing the other tasks.

One embodiment of this invention provides a method for wireless monitoring of esophageal pH value, based on the FIG. 5 and FIG. 7, comprising:
determining if the internal transmitting apparatus 30 locates in a preset region, through the cooperation between the recording apparatus 20 and the internal transmitting apparatus 30, and alerting if the internal transmitting apparatus 30 does not locate in the preset region; and
determining if the signal intensity is within a preset range, based on the intensity of the received signal, which is periodically detected by the external recording apparatus 20, and alerting if the signal intensity is not within the preset range.

The method, before the positioning system is put in use, further comprises:
pre-storing the first pH calibration data of the internal transmitting apparatus 30 in the external recording apparatus 20; and
calibrating the initial data received by the external recording apparatus 20 from the internal transmitting apparatus 30, and conducting temperature compensation in the course of calibration to obtain the current calibrated data;
comparing the current calibrated data with the first pH calibration data pre-stored, and sending the calibration alerting signal to the internal transmitting apparatus if the current calibrated data differ from the first pH calibration data; allowing the data recorder to alert through the buzzer 209 and/or the status indicator 208, under the control of the second micro-processor 201; and
controlling the internal transmitting apparatus to alert after the internal transmitting apparatus receives the calibration alerting signal.

The method for wireless monitoring of esophageal pH value is achieved by the cooperation between the first element 307 of the internal transmitting apparatus 30 and the second element of the external recording apparatus 20, which enables the real time monitoring of the position of the internal transmitting apparatus in the esophagus 1, not only avoids the invalid detection due to unexpected drop of the capsule, but also reduces the cost of detection for patients; in addition, the fact that the internal transmitting apparatus 30 is applied for detecting the intensity of the signal received may help to avoid signal interrupt problem caused when the patients are working or sleeping, which ensures the integrality of detection data; furthermore, the calibration of the system before being put in use and the temperature compensation in the course of calibration improve the testing accuracy.

It will be noted that the term “comprises/comprising” as used in this description is intended to denote the presence of a given characteristic, step or component, without excluding the presence of one or more other characteristic, features, integers, steps, components or groups thereof. While this invention has been described as having a preferred design, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

The invention claimed is:

1. A positioning system for wireless monitoring of esophageal pH value, comprising:
a) an internal transmitting apparatus (30); and
b) an external recording apparatus (20); wherein
said internal transmitting apparatus (30) comprises a pH sensor (301), a sample circuit (302), a first micro-processor (303), a power management unit (305), a first wireless transceiver module (304), and a first element (307);
said external recording apparatus (20) comprises a second micro-processor (201), a power management unit (210), a buzzer (209), a memory (205), a data interface (206), a key (207), a second wireless transceiver module (204), a status indicator (208), a housing (211), and a second element (203); and
said second element (203) of said external recording apparatus (20) cooperates with said first element (307) of said internal transmitting apparatus (30); if said external recording apparatus (20) detects that said internal transmitting apparatus (30) does not locate in a preset region, said micro-processor (201) controls said buzzer (209) and/or status indicator (208) to alert; and
said external recording apparatus (20) periodically detects the intensity of a signal received by said second wireless transceiver module (204), under the control of said micro-processor (201), and if the signal intensity is not within a preset range, said micro-processor (201) controls said buzzer (209) and/or status indicator (208) to alert.

2. The positioning system of claim 1, wherein
said first element (307) of said internal transmitting apparatus (30) is a permanent magnet;
said second element (203) of said external recording apparatus (20) is a magnetic sensor;
said second element (203) of said external recording apparatus (20) cooperates with said first element (307) of said internal transmitting apparatus (30); and
the intensity of magnetic field generated by said permanent magnet of said internal transmitting apparatus (30) is detected through said magnetic sensor, and if said intensity of magnetic field is not within the preset range, said external recording apparatus (20) detects that said internal transmitting apparatus (30) does not locate in the preset region.

3. The positioning system of claim 1, wherein
said first element (307) of said internal transmitting apparatus (30) is a reed switch disposed in series between said pH sensor (301) and said sample circuit (302);
said second element (203) of said external recording apparatus (20) is a magnet, which actuates said reed switch through magnetic induction;
said second element (203) of said external recording apparatus (20) cooperates with said first element (30) of said internal transmitting apparatus (30);
if the actual distance between said magnet and said reed switch is longer than a preset distance, said reed switch is actuated to open a circuit between said pH sensor (301) and said sample circuit (302) of said internal transmitting apparatus (30), and said external recording apparatus (20) detects that said internal transmitting apparatus (30) does not locate in the preset region.

4. The positioning system of claim 1, wherein
said external recording apparatus (20) further comprises a temperature sensor (202);
said memory (205) pre-stores a first pH calibration data of said internal transmitting apparatus (30);
said temperature sensor (202) detects a current room temperature, and sends the temperature data to said second micro-processor (201);
said second wireless transceiver module (204) receives an initial data from said internal transmitting apparatus (30) and sends the data to said second micro-processor (201);
said second micro-processor (201) calibrates said initial data, and conducts temperature compensation in the course of calibration, to obtain a current calibration data;
said second micro-processor (201) compares said current calibration data with said first pH calibration data pre-stored in said memory (205);
if said current calibration data differ from said first pH calibration data, a calibration alerting signal is sent to said internal transmitting apparatus (30) through second wireless transceiver module (204);
said internal transmitting apparatus (30) further comprises a work light (306);
said first wireless transceiver module (304) of said internal transmitting apparatus (30) receives said calibration alerting signal and sends the signal to said first micro-processor (303); and
said first micro-processor (303) controls said work light (306) to alert.

5. The positioning system of claim 2, wherein
said external recording apparatus (20) further comprises a temperature sensor (202);
said memory (205) pre-stores a first pH calibration data of said internal transmitting apparatus (30);
said temperature sensor (202) detects a current room temperature, and sends the temperature data to said second micro-processor (201);
said second wireless transceiver module (204) receives an initial data from said internal transmitting apparatus (30) and sends the data to said second micro-processor (201);
said second micro-processor (201) calibrates said initial data, and conducts temperature compensation in the course of calibration, to obtain a current calibration data;
said second micro-processor (201) compares said current calibration data with said first pH calibration data pre-stored in said memory (205);
if said current calibration data differ from said first pH calibration data, a calibration alerting signal is sent to said internal transmitting apparatus (30) through second wireless transceiver module (204);
said internal transmitting apparatus (30) further comprises a work light (306);
said first wireless transceiver module (304) of said internal transmitting apparatus (30) receives said calibration alerting signal and sends the signal to said first micro-processor (303); and
said first micro-processor (303) controls said work light (306) to alert.

6. The positioning system of claim 3, wherein
said external recording apparatus (20) further comprises a temperature sensor (202);
said memory (205) pre-stores a first pH calibration data of said internal transmitting apparatus (30);
said temperature sensor (202) detects a current room temperature, and sends the temperature data to said second micro-processor (201);
said second wireless transceiver module (204) receives an initial data from said internal transmitting apparatus (30) and sends the data to said second micro-processor (201);
said second micro-processor (201) calibrates said initial data, and conducts temperature compensation in the course of calibration, to obtain a current calibration data;
said second micro-processor (201) compares said current calibration data with said first pH calibration data pre-stored in said memory (205);
if said current calibration data differ from said first pH calibration data, a calibration alerting signal is sent to said internal transmitting apparatus (30) through second wireless transceiver module (204);
said internal transmitting apparatus (30) further comprises a work light (306);
said first wireless transceiver module (304) of said internal transmitting apparatus (30) receives said calibration alerting signal and sends the signal to said first micro-processor (303); and
said first micro-processor (303) controls said work light (306) to alert.

7. An internal transmitting apparatus, comprising:
a) a pH sensor (301);
b) a sample circuit (302);
c) a first micro-processor (303);
d) a power management unit (305);
e) a first wireless transceiver module (304); and
f) a first element (307);
wherein
said pH sensor (301), said sample circuit (302), said first micro-processor (303), and said first wireless transceiver module (304) are successively connected together;
said power management unit (305) is separately connected with said pH sensor (301), said sample circuit (302), said first micro-processor (303), and said first wireless transceiver module (304);
said sample circuit (302), said first micro-processor (303), said power management unit (305), and said first transceiver module (304) are enclosed in a capsule housing (308);
a sensing portion of said pH sensor (301) is exposed outside said capsule housing (308) to contact the body fluid in the esophagus; and
said first element (307) is disposed inside the capsule housing (308).

8. The internal transmitting apparatus of claim 7, wherein
said first element (307) is a permanent magnet, or a reed switch disposed in series between said pH sensor (301) and said sample circuit (302).

9. The internal transmitting apparatus of claim 7, further comprising a work light (306) connected with said first micro-processor (303) and receiving a control signal from said first micro-processor (303) to alert.

10. An external recording apparatus, comprising:
a) a second micro-processor (201);
b) a power management unit (210);
c) a buzzer (209);
d) a memory (205);
e) a data interface (206);
f) a key (207);
g) a second wireless transceiver module (204);
h) a status indicator (208);
i) a housing (211); and
j) a second element (203);
wherein
said second element (203) cooperates with a first element (307) of an internal transmitting apparatus (30);
if said internal transmitting apparatus (30) does not locate in a preset region, said second micro-processor (201) controls said buzzer (209) and/or said status indicator (208) to alert;
said external recording apparatus (20) periodically detects the intensity of a signal received by said second transceiver module (204) under the control of said second micro-processor (201), and if the signal intensity is not within a preset range, said second micro-processor (201) controls said buzzer (209) and/or said status indicator (208) to alert.

11. The external recording apparatus of claim 10, further comprising a temperature sensor (202), wherein
said memory (205) pre-stores a first pH calibration data of the internal transmitting apparatus (30);
said temperature sensor (202) detects a current room temperature, and sends the temperature data to said second micro-processor (201);
said second wireless transceiver module (204) receives an initial data from said internal transmitting apparatus (30) and sends the data to said second micro-processor (201);
said second micro-processor (201) calibrates said initial data, and conducts temperature compensation in the course of calibration, to obtain the current calibration data;
said second micro-processor (201) compares said current calibration data with said first pH calibration data pre-stored in said memory (205); and
if said current calibration data differ from said first pH calibration data, a calibration alerting signal is sent to said internal transmitting apparatus (30) through said second wireless transceiver module (204).

12. A method for wireless monitoring of esophageal pH value, comprising the steps of:
a) determining if an internal transmitting apparatus (30) locates in a preset region, through the cooperation between a recording apparatus (20) and said internal transmitting apparatus (30), and alerting if said internal transmitting apparatus (30) does not locate in the preset region; and
b) determining if the signal intensity is within a preset range, based on the intensity of a received signal, which is periodically detected by said external recording apparatus (20), and alerting if said signal intensity is not within the preset range.

13. The method of claim 12, prior to use, further comprising:
a) pre-storing a first pH calibration data of said internal transmitting apparatus (30) in said external recording apparatus (20); and
b) calibrating an initial data received by said external recording apparatus (20) from said internal transmitting apparatus (30), and conducting temperature compensation in the course of calibration on the initial data, to obtain a current calibrated data;
c) comparing said current calibrated data with said first pH calibration data pre-stored, and sending a calibration alerting signal to said internal transmitting apparatus (30) if said current calibrated data differ from said first pH calibration data; and
d) controlling said internal transmitting apparatus (30) to alert after said internal transmitting apparatus (30) receives said calibration alerting signal.
